(19) United States
(12) Patent Application Publication
(10) Pub. No.: US 2024/0371486 A1
(43) Pub. Date: Nov. 7, 2024

(54) METHODS AND SYSTEMS FOR PROGRAMMATIC CARE DECISIONING

(71) Applicant: Zachary Markin, Golden, CO (US)

(72) Inventor: Zachary Markin, Golden, CO (US)

(21) Appl. No.: 18/562,147

(22) PCT Filed: May 10, 2022
(86) PCT No.: PCT/US22/28531

§ 371 Date: Nov. 17, 2023

Related U.S. Application Data
(60) Provisional application No. 63/191,734, filed on May 21, 2021.

(51) Int. Cl. G16H 20/00; G16H 10/60; G16H 50/20

(52) U.S. Cl. CPC G16H 20/00; G16H 10/60; G16H 50/20

(57) ABSTRACT

Systems and methods for programmatic care decisioning are provided. In some aspects, the methods and systems determine one or more care decisioning inputs, one or more care decisioning logics, and one or more care conclusions. Provided are systems and methods for configuring programmatic medical care decisions, monitoring the accuracy of those care decisions when compared to human medical practitioners, and after evidence has accrued that the programmatic care decisions are sufficiently accurate, using that same programmatic logic to make care decisions. The systems and methods may be configured to surveil programmatic care decisions over time as the system is used and compare to traditional human driven decisions to ensure that the system is staying up to date with the current medical state of the art as practiced by human medical practitioners when faced with the same decision inputs.

### CROSS-REFERENCE TO RELATED APPLICATION

This application claims the priority benefit of U.S. Provisional Application No. 63/191,734, filed May 21, 2021, the entirety of which is incorporated herein by reference.

### BACKGROUND

Today, a patient may seek treatment for a given medical condition by being treated by a digital-first or in-person clinic. In such cases, the first step in treatment often comprises the patient completing a form to describe their symptoms and medical history. This process is cumbersome and can lead to errors. Improved methods and systems are needed.

### SUMMARY

It is to be understood that both the following general description and the following detailed description are exemplary and explanatory only and are not restrictive. In an embodiment, provided are methods and systems for a programmatic care decisioning tool as it relates to medical decisioning. The present methods and systems comprise a programmatic care decisioning tool which utilizes programmatic care decision making to determine and output one or more care conclusions.

The present methods and systems may be configured to provision patient care and programmatic decision-making. The present methods and systems present a programmatic care decisioning tool which utilizes models associated with medical decisions. For example, the medical decision may comprise a decision as to whether or not to prescribe a drug. The decision about whether or not to prescribe the drug is an example of a care decision that could be made by this system. Inputs to the system may include a patient supplied intake answer. A person skilled in the art will appreciate the inputs to the system may come from other sources as well. The system may be configured to apply one or more logical operations to the information gathered through the patient intake process alone or in conjunction with other external inputs. The result of these logical operations would be one of a number of care conclusions or an inconclusive result. For example, the system might result in three possible outcomes: “prescribe the drug”, “do not prescribe the drug”, or “inconclusive”. Because these programmatic care decisions will ultimately operate without human oversight, a configured care decision system might be subject to regulatory oversight by a body such as the FDA. As a result, the present methods and systems may also include a system for allowing a configured system to function in a “silent operation” mode. During this “silent operation” mode, care decisions are tracked but not used or shown to human medical practitioners. At the same time, the actual decision made by the human medical practitioner is stored as well as the inputs to that decision. Over time this data can be used to justify to regulatory bodies such as the FDA that the configured system can be transitioned to “production operations” where it operates making real live care decisions without human oversight. This system may be configured to operate in “production operations” for a quantity of care decisions to be handled in the traditional fashion (e.g., made by a medical professional) while the system is silently in the background making and tracking the care decisions it would make and comparing those to the care decisions made by the human practitioners. This surveillance data can be used to ensure the ongoing accuracy and quality of programmatic care decisions made by the system.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example system:

FIG. 2 shows an example system:

FIG. 3 shows an example method:

FIG. 4 shows an example method:

FIG. 5 shows an example method:

FIG. 6 shows an example method; and

FIG. 7 shows an example computing environment in which the present methods and systems may be executed.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Methods and systems are described for a programmatic care decisioning tool (“PCDT”). The programmatic care decisioning tool may make use of statistical modeling and individual, patient-specific variables to determine a care decision logic. The present methods and systems may incorporate data stored in a database, such as patient data, electronic health records, (EHRs), care decision logic, and other information.

The PCDT may comprise one or more graphical user interfaces (GUIs) and/or one or more application programming interfaces (APIs). The PCDT interfaces may be accessible via the internet, within a network, or embedded in a software program entirely. The PCDT may receive inputs and transmit or display outputs. For example, the tool may receive inputs such as patient data. Such inputs may include responses to surveys, diagnostic questions, demographic data, patient predictor variables, risk models, population data, and the like. Such inputs may be processed by a processor as described herein to generate outputs. Outputs may comprise a care conclusion. The outputs may be displayed on an interface. The interface may be configured to receive user inputs (e.g., inputs through, for example, a touch sensitive display as opposed to inputs fed to a processor from a database). The clinical programmatic care decisioning tool is a valuable tool to operationalize automated medical decision making based on logical operations. The inputs to these logical operations may be the outputs from traditional medical decision support tools, trained artificial intelligence or machine learning models or simple factual context. The programmatic care decisioning tool may be web based. The clinical programmatic care decisioning tool may autonomously operate a decision process for the care of one or more individuals.

The PCDT may facilitate shared decision making (SDM) in an automated and operationalized way by performing logical operations based on different variables and information supplied by the patient.

The tool may comprise a web-based tool such as that located at a URL. In another embodiment, the tool may comprise an application housed on a user device, for instance a tablet, laptop, smartphone, computer, or the like. Users may navigate the tool by any means including via a mouse, touchpad, keystroke input, voice input, touch input or the like or combinations thereof.

The tool may comprise an auto-population feature. The auto-population feature may interface with various databases (e.g. EHR databases, VASQIP, or CDW or the like), in order to retrieve relevant information (e.g., supplemental patient data such as medical histories, demographic data, combinations thereof, and the like). Fields may be populated via a user input or they may be auto-populated by, for example, querying a database. For example, demographic data and medical histories may be populated by pulling medical information from, for example, and electronic health record database.

A web-based and user-friendly portal may facilitate easy global access and use. Physicians and other clinicians may access the portal and enter data. As used throughout, a decision framework may refer to a formalized structure of a decision process. Inputs may comprise a set of information configured to inform a decision. For example, inputs may comprise patient data. The inputs may be associated with an input mechanism such as a survey, questionnaire, electronic health record (EHR), journal entries and other types of recorded information. A decision context may refer to all of the information that is incorporated and processed within a decision framework to reach a care conclusion. The decision context may include both specific inputs about a given decision as well as other relevant information which might be germane to a given decision. The broader decision context beyond specific inputs for a decision might be background information including factual constants (e.g. the normal range for a given laboratory diagnostic) or dynamic context specific to a given decision input (e.g. the current ICD-10 diagnostic codes associated with a patient's health record). Decision logic or care decision logic may refer to the set of logical operations which are applied to a decision context in order to reach a care conclusion. A care conclusion may refer to a final result of a decision framework resulting from the application of the decision logic to the decision context. A care conclusion set may refer to a group of valid conclusions which might result in a given decision framework. Dimensionality may refer to the nature of a value in a given decision framework. Dimensionality is the spatial, temporal, massive, or energetic characteristics (or some product of those things) of an element of the input set or a conclusion.

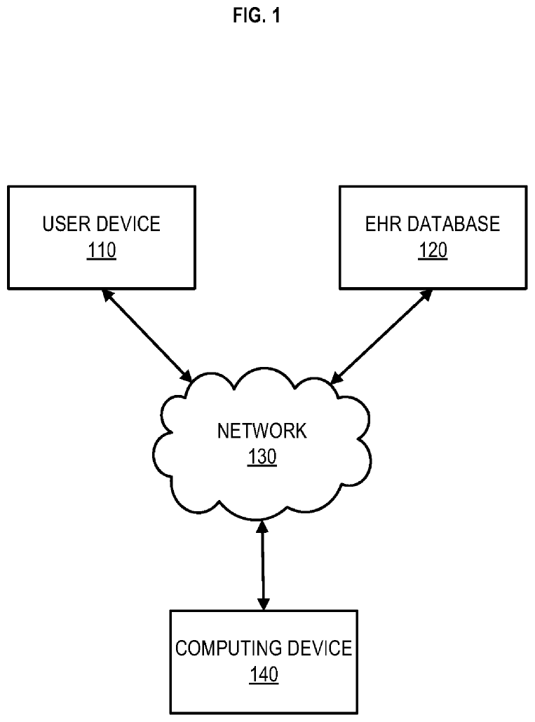

FIG. 1 shows an example system 100. The system 100 may comprise a user device 110, an EHR database 120, a network 130, and a computing device 140. The user device 110, the EHR database 120, and the computing device 140 may be configured to send and receive data via the network 130. The network 130 may comprise any telecommunications network such as the Internet or a local area network. Other forms of communications can be used such as wired or wireless telecommunication channels, for example. The network 130 may be an optical fiber network, a coaxial cable network, a hybrid fiber-coaxial network, a wireless network, a satellite system, a direct broadcast system, an Ethernet network, a high-definition multimedia interface network, a Universal Serial Bus (USB) network, or any combination thereof.

The user device 110 may comprise, for example, a smartphone, a tablet, a computer, combinations thereof, and the like. The user device 110 may comprise an interface configured to receive an input from a user (e.g., a “patient”). For example, the user device may be configured to present, via a display, a fillable form. The fillable form may comprise one or more selectable options. The fillable form may comprise, for example, a survey or questionnaire. For example, the fillable form may comprise a series of questions. The series of questions may be associated with a condition or diagnosis. The aforementioned example referring to the fillable form is merely exemplary and explanatory. A person skilled in the art will appreciate that the user device 110 may be configured to receive and/or determine the patient data via any means. For example, the user device may comprise a physician's tablet configured to receive and store medical case notes recorded in an ad-hoc manner (e.g., not a fillable form). For example, the user device 110 may be configured to receive one or more user inputs and determine, based on the one or more user inputs, one or more care decisioning inputs. The user device 110 may be configured to send, to the computing device 140, via the network 130, the one or more care decisioning inputs.

The system 100 may comprise an EHR database 120. The EHR database 120 may be configured to store one or more medical records. A medical record of the one or more medical records may be associated with the patient. The medical record may comprise supplemental patient data. For example, the medical record may comprise an identifier such as a name, address, telephone number, unique numerical identifier, combinations thereof, and the like. Supplemental patient data may comprise information associated with the patient, but not necessarily information included in or determined based on the fillable form. For example, if the fillable form may be associated with particular condition such as diabetes or hair loss while the supplemental patient data may comprise demographic data such as age, height, weight, region of residence, combinations thereof, and the like.

The computing device 140 may be configured to receive the primary patient data and/or the supplemental patient data, and/or the one or more care decisioning inputs from the user device 110 and/or the EHR database 120. Likewise, the computing device 140 may be configured to receive the supplemental data from the user device 110 and/or the EHR database 120. The computing device 140 may be configured to determine a care decision logic. The computing device 140 may determine the care decision logic based on the one more care decisioning inputs received from the user device 110 and/or the EHR database 120. Determining the care decision logic may comprise selecting, from a plurality of care decision logics, the care decision logic. The computing device 140 may be configured to select the care decision logic based on the primary patient data and/or the supplemental patient data. For example, the primary patient data may indicate a condition. The condition may be associated with medical diagnosis. For example, the primary patient data may be associated with a fillable form comprising questions related to diabetes, hair loss, erectile dysfunction, or any other medical diagnosis. The aforementioned are merely exemplary and explanatory and a person skilled in the art will appreciate that the methods and systems described herein may be applied to any condition and/or diagnosis and will further understand that the terms “condition” and “diagnosis” are similar and may be used interchangeably. The computing device 140 may be configured to determine the care decision logic based on the primary patient data, the supplemental patient data, and/or metadata associated with either the primary patient data and/or the supplemental patient data. For example, the primary patient data (e.g., the populated fillable form), may comprise or otherwise be associated with a form identifier. The form identifier may be associated with the condition. For example, a first fillable form of a plurality of fillable forms may be associated with a first identifier 00001A and a second fillable form of the plurality of fillable forms may be associated with a second identifier 00002B. Upon receipt of the fillable form, the computing device 140 may determine the first identifier and may determine, based on the first identifier that the first fillable form is associated with a diabetes questionnaire. Similarly, upon receipt of the fillable form, the computing device 140 may determine the second identifier and may determine, based on the second identifier, that the fillable form is associated with hair loss. The computing device 140 may select the care decision logic based on determining the condition associated with the fillable form. For example, based on determining that the received input comprises a fillable form associated with diabetes, the computing device 140 may select a care decision logic associated with diabetes. Similarly, the computing device 140 may select a care decision logic associated with hair loss if the computing device 140 determines the received input is associated with hair loss.

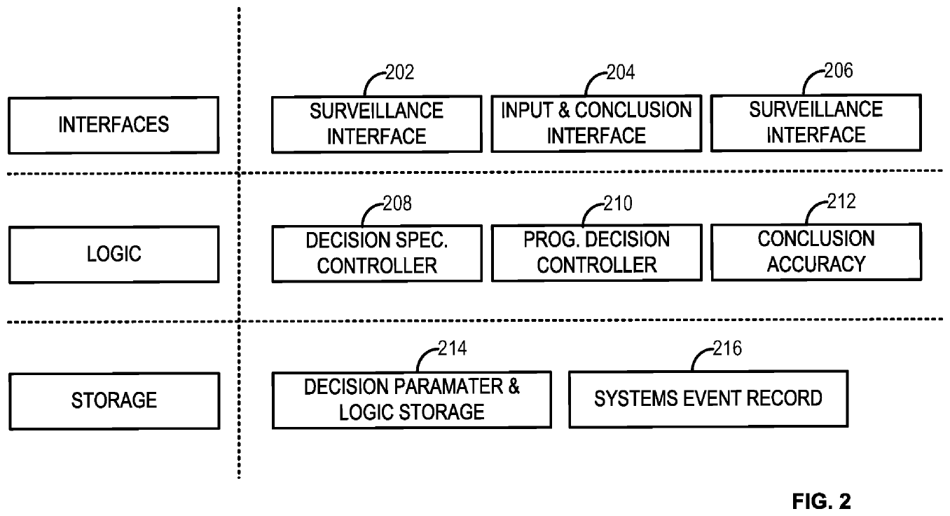

FIG. 2 shows an example system 200. The system 200 may comprise one or more interfaces, one or more logic modules, and one or more storage modules. For example, the one or more interfaces may comprise a framework specification interface 202, an input and conclusion interface 204, and a surveillance interface 206. The one or more interfaces may make up a subsystem comprising system components which users or external systems interact with the system. These interfaces may be programmatic or graphical in nature. These interfaces may receive inputs from external systems or users and output responses to those external systems or users. The framework specification interface 202 may comprise an interface wherein users of the system specify the necessary inputs and decision context for a given decision framework as well as the possibility set of resulting decisions. This interface also enables specification of the logic necessary to reach one of the possible decision outputs. The input and conclusion interface 204 may comprise an interface configured for inputting the specific context necessary for the system to reach a conclusion. This interface may to the user or external system that provided the context for a decision and supplies the conclusion reached. The surveillance interface may comprise an interface configured to provide users or external systems the ability to monitor when a given decision logic has been proven to a statistically meaningful degree that it can be used instead of a human for making that decision in actual care. The surveillance interface may also be configured for monitoring the ongoing fidelity of conclusions reached by the system compared to conclusions reached by human medical practitioners when provided the same input context.

The one or more logic modules may comprise a decision specification controller 208, a programmatic decision controller 210, and a conclusion accuracy module 212. The one or more logic modules may make up system a comprising logical components which control the system, receive inputs from the interface subsystem, process inputs and stored data, and produce output to be sent via the interfaces to external users or systems. The decision specification controller (DSC) module 208 may be configured to accept decision specifications including possible decision inputs and decision conclusions as well as processing instructions from the framework specification interface. The DCS may be configured to process these inputs and may be further configured to write to the storage subsystem. Thus, these specifications can be used later to logically drive decisions.

The programmatic decision controller 210 may be configured to process a given decision context and determine a conclusion. For example, the programmatic decision controller 210 may be configured to determine a care decision logic and apply the care decision logic to patient data. For example, the programmatic decision controller may determine the patient data is associated with a condition (e.g., diabetes, hair loss, erectile dysfunction, etc.). The programmatic decision controller may determine the patient data is associated with the condition by virtue of, for example, an identifier associated with the patient data. For example, the patient data may be received via a questionnaire. The questionnaire may comprise metadata (e.g., one or more identifiers) indicating the condition. For example, the questionnaire may be associated with an ICD code indicating the received patent data is related to a potential diagnosis. The metadata may be used to select a care decision logic associated with that identifier.

The decision accuracy logic module 212 may be configured to observe conclusions reached by one or more human medical practitioners and send them to the system event record for storage. The decision accuracy logic may process both system produced conclusions and records of conclusions produced by human medical practitioners to determine when a decision logic has been proven to be programmatically equivalent or better than conclusions reached by human medical practitioners in a statistically significant way. The decision accuracy logic also processes a testing sample of decisions after the system is operational so that a deviation between the programmatic and human conclusions can be detected if it starts to emerge.

The one or more storage modules may comprise a decision parameter and logic storage 214 and a systems event record module 216. The one or more storage modules may make up a subsystem comprising storage of decision input dimensionality and ranges, logical operations for a given decision, and a record of all system conclusions and decision contexts, as well as conclusions reached by human medical practitioners and their corresponding decision contexts. The decision parameter and logic storage 214 may be configured to store the dimensions and scope of valid inputs for a given care decision, the possible outputs or conclusions which can be reached, and the operations to be performed when the system is computing decision inputs to reach a programmatic care conclusion. The system event record module 216 may be configured to store all of the input contexts and corresponding conclusions the system has reached as well as any human medical practitioner decisions which were observed by the system.

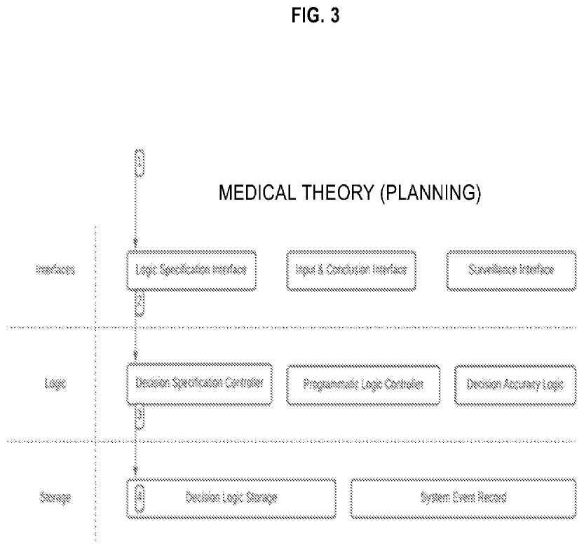

FIG. 3 shows an example method 300. The method 300 may be associated with a medical planning process flow. During a planning phase, a user of the system may plan a decision framework based on knowledge of medical protocols. That is to say, during the planning phase, one or more medical practitioners may create a logic based medical decision framework and articulate the necessary inputs and range of valid conclusions for that decision. A practitioner may articulate how the inputs should be processed in order to reach a medical conclusion. When planning a decision framework, a user may describe the decision context which is made of the types of inputs and relevant contextual information that go into a decision as well as the valid ranges of input and contextual information. Secondly, the user describes how this decision context is processed to reach a conclusion. Thirdly, the user describes the set of valid conclusions which can be reached for this decision. During configuration, the practitioner may interact with the decision specification interface to record in the system the necessary inputs, range of conclusions, and logic for processing inputs to reach the conclusion.

For example, clinical configuration may be carried out by a medical professional. For example, when determining the care decision logic (e.g., “planning” the care decision logic), a medical professional may determine questions to be answered, fields to be populated, etc. For example, in order to generate or plan a diabetes care decision logic, the input mechanism may be configured to receive answers related to blood glucose values and timing data. For example, a diabetes questionnaire may be configured to receive patient inputs associated with various historical blood glucose levels and their time of occurrence. In this case, a first dimension of the input may comprise a blood glucose value (e.g., as measured in milligrams per deciliter). Similarly, a second dimension of the input patient data may comprise a time value (e.g., a date and time at which the blood glucose value was determined).

At step 301, the user may describe, via the Logic Specification Interface, the decision context, the logical operations to be performed on the decision context to reach a conclusion, the set of valid decision conclusions, as well as the dimensionality and valid range of the decision context and possible conclusions.

At step 302, the Logic Specification Interface may send the decision context, decision logic, conclusion set, and the dimensionality of decision context and conclusion set to the Decision Specification Controller.

At step 303, the Decision Specification Controller may write the decision context, decision logic, conclusion set, and the dimensionality of decision context and conclusion to the Decision Logic Storage where it creates a persistent record of each of those items to be stored.

While this description shows these steps happening fully sequentially (the user describes the full decision context, processing, logic, and conclusions which is then followed by processing and storage), it is to be understood that some subset of this information such as the decision context or part of the decision context might be entered, processed, and saved before another subset of this information might be entered, processed, and saved.

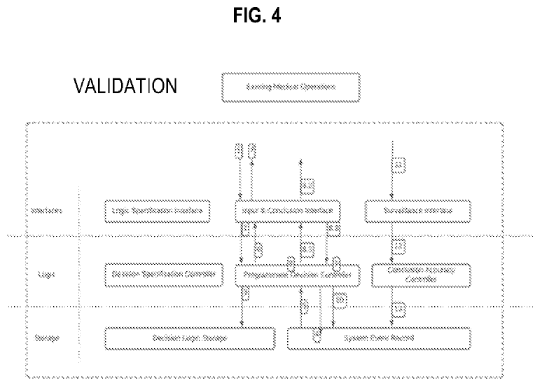

FIG. 4 shows an example method 400. The method 400 may be associated with a validation process flow. During the validation process flow, the system may operate in the background, receiving inputs for decisions and reaching conclusions. During this time human medical practitioners are also practicing as they normally would and the conclusions which they reach are also input to the system via the input and conclusion interface. While the below steps are described in an order, it is to be understood they may be performed in any order.

At step 401, one or more decision inputs and context along with the corresponding decision framework may be sent to (or otherwise provided to) the Input & Conclusion Interface. The Input & Conclusion Interface may be configured to receive the one or more decision inputs, the context, and the corresponding decision framework. For example, the one or more decision inputs may comprise a set of information configured to inform a decision. For example, inputs may comprise patient data. The inputs may be associated with an input mechanism such as a survey, questionnaire, electronic health record (EHR), journal entries and other types of recorded information. A decision context may refer to all of the information that is incorporated and processed within a decision framework to reach a care conclusion. The decision context may include both specific inputs about a given decision as well as other relevant information which might be germane to a given decision. The broader decision context beyond specific inputs for a decision might be background information including factual constants (e.g. the normal range for a given laboratory diagnostic) or dynamic context specific to a given decision input (e.g. the current ICD-10 diagnostic codes associated with a patient's health record).

The decision context may refer to all of the information that is incorporated and processed within a decision framework to reach a care conclusion. The decision context may include both specific inputs about a given decision as well as other relevant information which might be germane to a given decision. The broader decision context beyond specific inputs for a decision might be background information including factual constants (e.g. the normal range for a given laboratory diagnostic) or dynamic context specific to a given decision input (e.g. the current ICD-10 diagnostic codes associated with a patient's health record). Decision logic or care decision logic may refer to the set of logical operations which are applied to a decision context in order to reach a care conclusion.

The decision framework may comprise a formalized structure of a decision process.

At step 402, the Input & Conclusion Interface may provide the one or more decision inputs, the context, and the corresponding decision framework to the Programmatic Decision Controller. The programmatic decision controller may be configured to receive the one or more decision inputs, the context, and the corresponding decision framework.

At step 403, the Programmatic Decision Controller may be configured to determine one or more Decision Framework Parameters from the Decision Logic Storage. The Programmatic Decision Controller may be further configured to determine a logic. For example, the Programmatic Decision Controller may be configured to query the Decision Logic Storage to determine the logic. Further, the Programmatic Decision Controller may be configured to determine a unique identifier (UID) for the particular Decision Framework being processed.

At step 404, the Programmatic Decision Controller may determine and/or generate a Decision Instance of the given Decision Framework by writing a new Decision Instance to the System Event Record. For example, writing the new Decision Instance may comprise sending or receiving data to or from database. The data may cause the database to store the Decision Instance. This Decision Instance may be instantiated with the Inputs and Decision Context provided to the Input & Conclusion Interface as well as the UID corresponding to the Decision Framework.

At step 405, at creation of the Decision Instances, a UID may be created for that Decision Instance. For example, the UID may be associated with the Decision Instance. Associating the UID and the Decision Instance may comprise associating the UID and the Decision Instance in a database. For example, a first field of a plurality of fields may be populated with the UID while a second field of the plurality of fields may be populated with the Decision Instance. This decision instance UID may be passed back to the programmatic decision controller.

At step 406, the programmatic decision controller may be configured to receive the decision instance UID. The programmatic decision controller may send the decision instance UID back to the input & conclusion interface.

At step 407, the input and conclusion interface may be configured to receive the decision instance UID. The input and conclusion interface may be further configured to respond to the original inbound decision input and context with the UID of the Decision Instance.

At step 408, the Programmatic Decision Controller may process the inputs, decision context, and decision framework parameters.

Optionally, at step 408.1, the programmatic decision controller via the Input & Conclusion Interface may make external lookups to collect dynamic decision input information if specified and is the result of programmatic decision logic. For example, the programmatic decision controller, via the input and conclusion interface, may query one or more external databases.

Optionally, at step 408.2, the Input and Conclusion Interface may make an external lookup by, for example, querying the one or more external databases.

Optionally, at step 408.3, the Input and Conclusion Interface may send this externally identified value back to the programmatic decision controller. The programmatic decision controller may be configured to receive the identified value.

At step 409, the Programmatic Decision Controller may be configured to execute a logical processing and determine a “Conclusion” for the given Decision Instance. The conclusion may comprise a final result of a decision framework resulting from the application of the decision logic to the decision context. The care conclusion may be determined based on a care conclusion set. The care conclusion set may comprise one or more of a plurality of valid conclusions which may result in a given decision framework.

At step 410, the Programmatic Logic Controller may write the Conclusion to the System Event Record. For example, the Programmatic Logic Controller may record the Conclusion in the System Event Record. The Conclusion recorded in the System Event Record may be associated with the Decision Instance by reference to the Decision Instance UID and thereby the corresponding Decision Framework UID.

At step 411, the Surveillance Interface may receive the Conclusion reached by the human practitioner or a previously validated programmatic care decision framework which is known to perform as well or better than human practitioners. This Conclusion reached may be provided together with the Decision Instance UID that it was associated with.

At step 412, the Surveillance Interface may send this Conclusion reached by the human practitioner or a Conclusion reached by another previously validated programmatic care decision framework together with the Decision Instance UID to the Conclusion Accuracy Controller. The Conclusion Accuracy Controller may be configured to receive the Conclusion reached by the human practitioner.

At step 413, the Conclusion Accuracy Controller may write the actual Conclusion reached by the human practitioner to the System Event Record together with the corresponding Decision Instance UID.

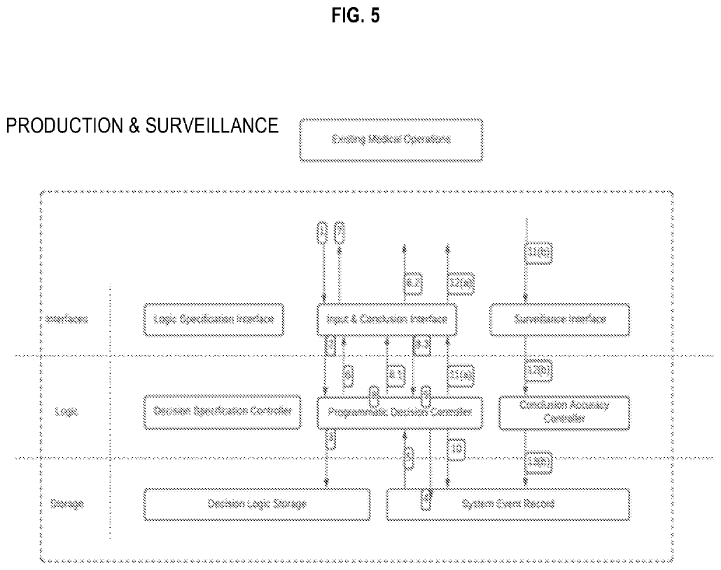

FIG. 5 shows an example method 500. The method 500 may be associated with a production and surveillance phase. During production and surveillance phase, the system may make a portion of (e.g., most, a majority, all) of actual care decisions. Some may be made by human medical practitioners (or previously validated programmatic decision frameworks). A previously validated programmatic decision framework may be a decision framework which has already been demonstrated to have an error rate falling below a threshold. This fraction of decisions made by human medical practitioners are used for purposes of system surveillance to ensure the quality of conclusions reached by the system and fidelity of those conclusions to those reached by human medical practitioners.

At step 501, decision inputs and context along with the corresponding decision framework are provided to Input & Conclusion Interface. The Input & Conclusion Interface may be configured to receive the one or more decision inputs, the context, and the corresponding decision framework. For example, the one or more decision inputs may comprise a set of information configured to inform a decision. For example, inputs may comprise patient data. The inputs may be associated with an input mechanism such as a survey, questionnaire, electronic health record (EHR), journal entries and other types of recorded information. A decision context may refer to all of the information that is incorporated and processed within a decision framework to reach a care conclusion. The decision context may include both specific inputs about a given decision as well as other relevant information which might be germane to a given decision. The broader decision context beyond specific inputs for a decision might be background information including factual constants (e.g. the normal range for a given laboratory diagnostic) or dynamic context specific to a given decision input (e.g. the current ICD-10 diagnostic codes associated with a patient's health record).

The decision context may refer to all of the information that is incorporated and processed within a decision framework to reach a care conclusion. The decision context may include both specific inputs about a given decision as well as other relevant information which might be germane to a given decision. The broader decision context beyond specific inputs for a decision might be background information including factual constants (e.g. the normal range for a given laboratory diagnostic) or dynamic context specific to a given decision input (e.g. the current ICD-10 diagnostic codes associated with a patient's health record). Decision logic or care decision logic may refer to the set of logical operations which are applied to a decision context in order to reach a care conclusion.

At step 502, the Input & Conclusion Interface may send the inputs, the context, and the framework to the Programmatic Decision Controller. The programmatic decision controller may be configured to receive the one or more decision inputs, the context, and the corresponding decision framework.

At step 503, the Programmatic Decision Controller may query the Decision Logic Storage to determine the one or more Decision Framework Parameters and logic from the Decision Logic Storage along with a unique identifier (UID) for this particular Decision Framework being processed.

At step 504, the Programmatic Decision Controller may generate a Decision Instance of the given Decision Framework by writing a new Decision Instance to the System Event Record. For example, writing the new Decision Instance may comprise sending or receiving data to or from database. The data may cause the database to store the Decision Instance. This Decision Instance is instantiated with the Inputs and Decision Context provided to the Input & Conclusion Interface as well as the UID corresponding to the Decision Framework.

At step 505, at creation of the Decision Instances, a UID is created for that Decision Instance. This decision instance UID is passed back to the programmatic decision controller. For example, the UID may be associated with the Decision Instance. Associating the UID and the Decision Instance may comprise associating the UID and the Decision Instance in a database. For example, a first field of a plurality of fields may be populated with the UID while a second field of the plurality of fields may be populated with the Decision Instance.

At step 506 the programmatic decision controller may be configured to receive the decision instance UID. The programmatic decision controller may send the decision instance UID back to the input & conclusion interface.

At step 507, the input and conclusion interface may be configured to receive the decision instance UID. The input and conclusion interface may be further configured to respond to the original inbound decision input and context with the UID of the Decision Instance.

At step 508, the Programmatic Decision Controller processes inputs, decision context, and decision framework parameters.

Optionally, at step 508.1, the programmatic decision controller via the Input & Conclusion Interface may make external lookups to collect dynamic decision input information if specified and is the result of programmatic decision logic. For example, the programmatic decision controller, via the input and conclusion interface, may query one or more external databases.

Optionally, at step 508.2, the Input and Conclusion Interface may make an external lookup. For example, the Input and Conclusion Interface may query an external database to determine a value.

Optionally, at step 508.3, the Input and Conclusion Interface might pass this externally identified value back to the programmatic decision controller. The programmatic decision controller may be configured to receive the identified value.

At step 509, the Programmatic Decision Controller may execute a logical processing. The Programmatic Decision Controller may determine a “Conclusion” for the given Decision Instance.

At step 510, the Programmatic Logic Controller may write the Conclusion reached to the System Event Record. The Conclusion recorded in the System Event Record may be associated with the Decision Instance by reference to the Decision Instance UID and thereby the corresponding Decision Framework UID.

Steps 511 (a) and 512 (a) may be utilized when the system is in production operation and making active care decisions autonomously.

Optionally, at step 511 (a), the programmatic decision controller may send the Conclusion back to the Input & Conclusion Interface.

Optionally, at step 512 (a), the Input & Conclusion Interface may receive the Conclusion. The Input & Conclusion Interface may be configured to respond to the original input request with the care decision conclusion.

Steps 511 (b), 512 (b), and 513 (b) may be utilized when the system is in production operation but the decision is being surveilled for quality assurance purposes and compared to the decision made by the human practitioner or to decision made by previously validated programmatic care decision frameworks. These steps are similar as those described with respect to method 400.

Optionally, at step 511 (b), the Surveillance Interface may receive the Conclusion reached by the human practitioner. This Conclusion reached may be provided together with the Decision Instance UID that it was associated with.

Optionally, at step 512 (b), the Surveillance Interface passes this Conclusion reached by the human practitioner together with the Decision Instance UID to the Conclusion Accuracy Controller.

Optionally, at step 513 (b), the Conclusion Accuracy Controller may receive the Conclusion and the Decision Instance UID. The Conclusion Accuracy Controller may write the actual Conclusion reached by the human practitioner to the System Event Record together with the corresponding Decision Instance UID.

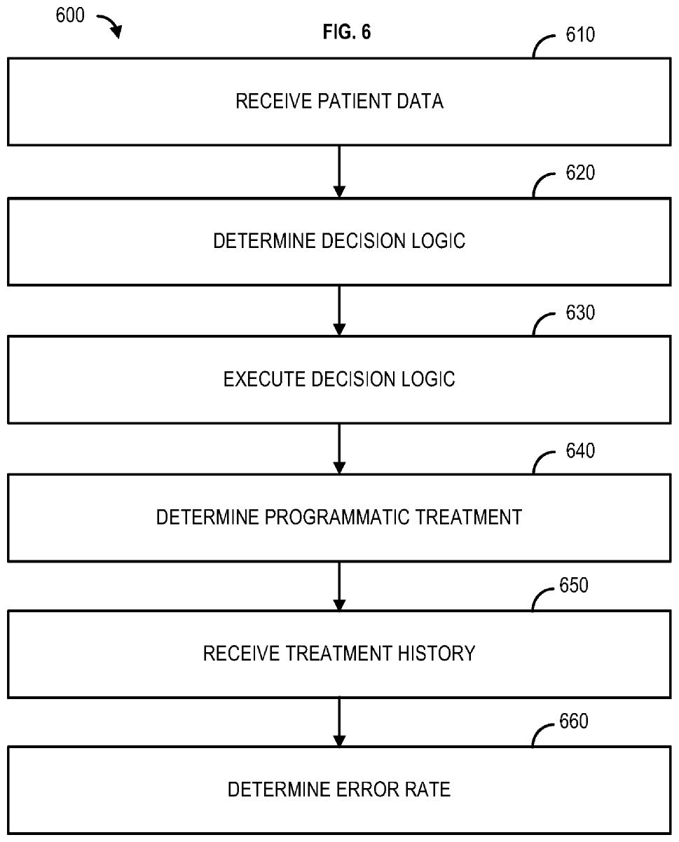

FIG. 6 shows an example method 600. The method 600 may be executed by any one or more of the devices described herein. At step 610, patient data may be received. The patient data may comprise primary patient data and supplementary patient data. The primary patient data may comprise diagnostic data. For example, for the diagnostic data may comprise data indicative of one or more medical condition such as diabetes, hair loss, or erectile dysfunction. The aforementioned diagnosis are merely exemplary and explanatory and a person skilled in the art will appreciate that any data indicative of any condition or diagnosis is contemplated by the methods and systems described here.

The supplementary patient data may comprise, for example, demographic data or other information associated with the patient but not, perhaps, directly related to (e.g., indicative of) a condition or diagnosis. The patient data may comprise an identifier. The patient data may be associated with a diagnostic identifier. For example, the diagnostic identifier may comprise an ICD-9 code. The aforementioned is exemplary and explanatory only and a person skilled in the art will appreciate that any identifier may be used.

Determining the patient data may comprise receiving the patient data from a user device. For example, the user device comprise an interface configured to receive one or more user inputs. The one or more user inputs may comprise, for example, answers to survey questions and/or diagnostic questions. For example, the user device may display a questionnaire configured to determine whether a user is suffering from hair loss. The questions may be part of a standard diagnostic protocol. The questionnaire may comprise selectable options, fillable fields, and other mechanisms configured to receive the one or more user inputs. Similarly, the patient data may be determined based on a query to a database. For example, a computing device (e.g., the user device) may query a database storing EHRs to determine the patient data. The aforementioned are merely exemplary and explanatory and a person skilled in the art will appreciate that the patient data can comprise any information and may be determined according to any known technique.

At step 620, a care decision logic may be determined. The care decision logic may be determined based on the patient data. For example, the patient data may be associated with an identifier. The identifier may be associated with a condition or diagnosis. For example, the identifier may be associated with an ICD-10 code or other diagnostic code. The care decision logic may be determined based on a format of the patient data. For example, the patient data may be received in the form of a questionnaire. For example, the questionnaire may comprise a “fill-in-the-bubble” type questionnaire. If the patient data is received in the form of a questionnaire, optical analysis may be implemented to determine a plurality of questions and a plurality of answers. For example, a computing device may be configured to determine a pattern of bubbles. The pattern of bubbles may indicate the plurality of answers. Based on the pattern of bubbles, the computing device may be configured to determine the patient data.

In the case the patient data is text, the computing device may perform OCR or other similar techniques and transcribe the patient data into a computer readable format. Similarly, if the patient data is audio data (e.g., speech describing symptoms, slurred speech as evidence of a problem, sounds like coughing, or other noises), the computing device may perform natural language processing/natural language understanding (NLP/NLU) to convert the patient data to a computer readable format.

At step 630, the care decision logic may be executed. Executing the care decision logic may comprise applying the care decision logic to the patient data such as making a decision to prescribe a medication to the patient, enroll the patient in a therapy or rehabilitation program, direct a patient to receive additional diagnostics or consultations, or any other direction of the care for the patient. For example, in the case of a questionnaire, the patient data may comprise a plurality of answers to a plurality of questions. The plurality of answers may indicate the presence of a condition. For example, if the questionnaire comprises ten yes or no questions, the care decision logic may be configured to determine the answer to each of the ten yes or no questions and determine whether a subset of the answers satisfies a diagnostic threshold. For example, the questionnaire may be configured such that if the patient answers “yes” to all the even numbered questions and “no” to the all the odd numbered questions, the subset of “yes” answers may indicate the presence of diabetes, hair loss, erectile dysfunction, or any other condition or diagnosis. Similarly, if the input (e.g., the patient data) comprises a fillable form, the care decision logic may determine values associated with the populated fields of the fillable forms. For example, in the case of diabetes, fillable fields may comprise blood glucose values and timestamps. The care decision logic may determine that the blood glucose values and associated timestamps satisfy a diabetes diagnostic threshold. The aforementioned is merely exemplary and explanatory and a person skilled in the art will appreciate that any input may be used and the respective care decision logic is configured to determine the presence or absence of the condition or diagnosis. Determining the presence or absence of the condition may comprise determining and ICD-9 code associated with the condition. While ICD-9 is mentioned, that example should not be construed as limited and a person skilled in the art will appreciate that any identifier or code may be used. For example, the Type II diabetes mellitus ICD code is E11* while the erectile dysfunction ICD code is N52.9 while the hair loss ICD code is L65.9.

At step 640, one or more programmatic treatments may be determined. The one or more programmatic treatments may be associated with the condition. For example, the condition may be associated with an ICD-9 code. Likewise, the one or more programmatic treatments may be associated with the same ICD-9 code.

At step 650, a treatment history may be received. Receiving the treatment history may comprise querying a database for the treatment history and receiving, in response to the query, the treatment history. The treatment history may comprise a record of inputs (e.g., patient data) and care conclusion (e.g., diagnosis) associated with similar patient data. For example, if the patient answers all the even numbered questions with “yes,” the computing device may query the database to determine other instances where a similar input has been received and then determine associated historical care conclusions. For example, the treatment history may comprise one or more historical care conclusions associated with the condition. Further, the treatment history may comprise a record of therapies prescribed for the condition in the past.

At step 660, an error rate may be determined. The error rate may comprise w a difference between the one or more programmatic treatments and the one or more historical treatments The error rate may comprise a comparison between the programmatic treatment or care decisions and the historical care conclusions. For example, a record may be generated for each care conclusion and a determination made as to whether or not that care conclusion is in line with historical care conclusions. Conversely, an accuracy rate may be determined.

The method may further comprise determining the error rate satisfies or fails to satisfy one or more thresholds. The one or more error rate thresholds may be any value or quantity (e.g., 50% error rate threshold, 90% accuracy rate threshold, 99% accuracy rate threshold, or the like). The method may further comprise outputting, based on determining the error rate and/or accuracy rate satisfies or fails to satisfy the one or more thresholds, a message. The message may indicate the error rate and/or accuracy rate satisfies or fails to satisfy the one or more thresholds. The method may further comprise outputting the care conflusion.

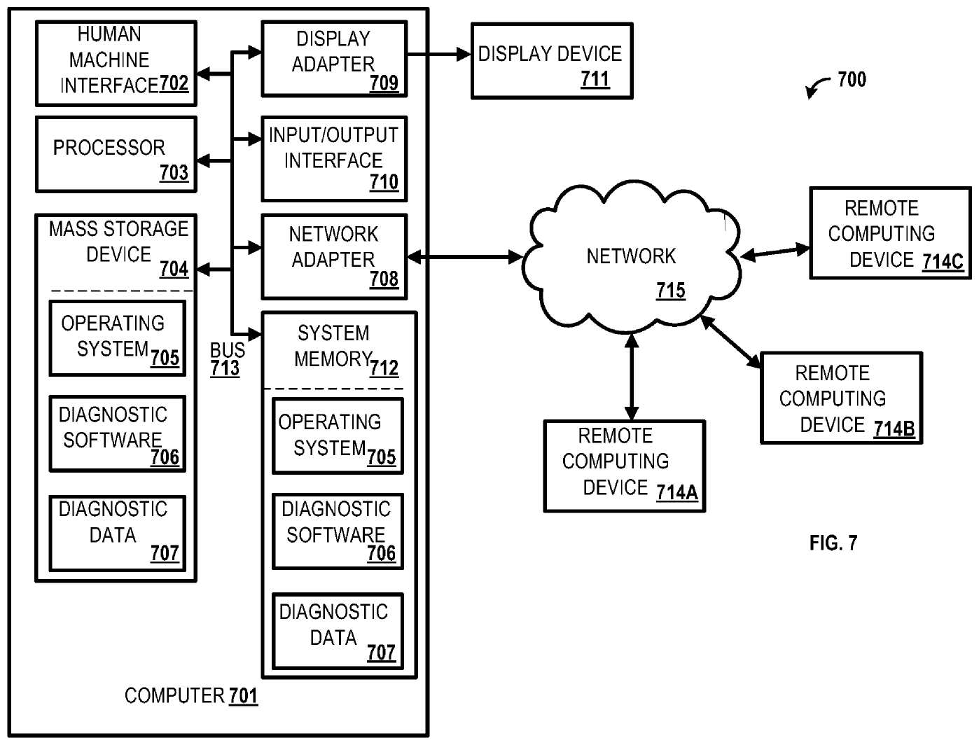

FIG. 7 is a block diagram illustrating an exemplary operating environment 700 for performing the methods described herein. In an exemplary example, the methods and systems of the present description can be implemented on a computer 701 as illustrated in FIG. 7 and described below. By way of example, each of the devices of FIG. 1 may be a computer 701 as illustrated in FIG. 7. Similarly, the methods and systems described can utilize one or more computing devices to perform one or more functions in one or more locations. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and/or the like.

The processing of the described methods and systems can be performed by software components. The described systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The described methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods described herein can be implemented via a general-purpose computing device in the form of a computer 701. The components of the computer 701 can comprise, but are not limited to, one or more processors 703, a system memory 712, and a system bus 713 that couples various system components including the processor 703 to the system memory 712. In the case of multiple processors 703, the system can utilize parallel computing.

The system bus 713 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 713, and all buses specified in this description can also be implemented over a wired or wireless network connection and each of the subsystems, including the processor 703, a mass storage device 704, an operating system 705, diagnostic software 706, diagnostic data 707, a network adapter 717, system memory 712, an Input/Output Interface 710, a display adapter 709, a display device 711, and a human machine interface 702, can be contained within one or more remote computing devices 714 A,B,C at physically separate locations, connected through buses of this form, in effect implementing a fully distributed system.

The computer 701 typically includes a variety of computer readable media. Exemplary readable media can be any available media that is accessible by the computer 701 and includes, for example and not meant to be limiting, both volatile and non-volatile media, removable and non-removable media. The system memory 712 includes computer readable media in the form of volatile memory, such as random access memory (RAM), and/or non-volatile memory, such as read only memory (ROM). The system memory 712 typically contains data, such as network data 707, and/or program modules, such as operating system 705 and diagnostic software 706, that are immediately accessible to and/or are presently operated on by the processor 703.

In another example, the computer 701 can also comprise other removable/non-removable, volatile/non-volatile computer storage media. By way of example, FIG. 7 illustrates a mass storage device 704 which can provide non-volatile storage of computer code, computer readable instructions, data structures, program modules, and other data for the computer 701. For example and not meant to be limiting, a mass storage device 704 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 704, including by way of example, an operating system 705 and diagnostic software 706 (e.g., to encrypt/decrypt network credentials, generate a network, send/receive data to/from an access point, etc.). Each of the operating system 705 and diagnostic software 706 (or some combination thereof) can comprise elements of the programming and the diagnostic software 706. The diagnostic data 707 (e.g., patient data, care conclusions, diagnostic histories, population histories, combinations thereof, and the like) can also be stored on the mass storage device 704. The diagnostic data 707 can be stored in any of one or more databases known in the art. Examples of such databases comprise, DB2 R, Microsoft R: Access, Microsoft R: SQL Server, Oracle®, mySQL, PostgreSQL, and the like. The databases can be centralized or distributed across multiple systems.

In another example, the user can enter commands and information into the computer 701 via an input device (not shown). Examples of such input devices comprise, but are not limited to, a keyboard, pointing device (e.g., a “mouse”), a microphone, a joystick, a scanner, tactile input devices, such as gloves, and other body coverings, and the like These and other input devices can be connected to the processor 703 via a human machine interface 702 that is coupled to the system bus 713, but can be connected by other interface and bus structures, such as a parallel port, game port, an IEEE 1374 Port (also known as a Firewire port), a serial port, or a universal serial bus (USB).

In yet another example, a display device 711 can also be connected to the system bus 713 via an interface, such as a display adapter 709. It is contemplated that the computer 701 can have more than one display adapter 709 and the computer 701 can have more than one display device 711. For example, a display device can be a monitor, an LCD (Liquid Crystal Display), or a projector. In addition to the display device 711, other output peripheral devices can comprise components, such as speakers (not shown) and a printer (not shown) which can be connected to the computer 701 via Input/Output Interface 710. Any step and/or result of the methods can be output in any form to an output device. Such output can be any form of visual representation, including, but not limited to, textual, graphical, animation, audio, tactile, and the like. The display 711 and computer 701 can be part of one device, or separate devices.

The computer 701 can operate in a networked environment using logical connections to one or more remote computing devices 714A,B,C. By way of example, a remote computing device can be a personal computer, portable computer, smartphone, a server, a router, a network computer, a peer device or other common network node, and so on. Logical connections between the computer 701 and a remote computing device 714 A,B,C can be made via a network 715, such as a local area network (LAN) and/or a general wide area network (WAN). Such network connections can be through a network adapter 717. A network adapter 717 can be implemented in both wired and wireless environments. Such networking environments are conventional and commonplace in dwellings, offices, enterprise-wide computer networks, intranets, and the Internet.

For purposes of illustration, application programs and other executable program components, such as the operating system 705 are illustrated herein as discrete blocks, although it is recognized that such programs and components reside at various times in different storage components of the computing device 701, and are executed by the data processor(s) of the computer. An implementation of diagnostic software 706 can be stored on or transmitted across some form of computer readable media. Any of the described methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information, such as computer readable instructions, data structures, program modules, or other data. Exemplary computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

While the methods and systems have been described in connection with specific examples, it is not intended that the scope be limited to the particular embodiments set forth, as the embodiments herein are intended in all respects to be illustrative rather than restrictive. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation: the number or type of embodiments described in the specification.

It will be apparent to those skilled in the art that various modifications and variations can be made without departing from the scope or spirit. Other embodiments will be apparent to those skilled in the art from consideration of the specification and practice described herein. It is intended that the specification and examples be considered as exemplary only, with a true scope and spirit being indicated by the following claims.

What is claimed is:

1. A method comprising:
receiving, by a computing device, patient data;
determining, based on the patient data, a care decision logic;
executing, based on the patient data, the care decision logic to determine a care conclusion;
determining, based on executing the care decision logic and the care conclusion, one or more programmatic treatments;
receiving, by the computing device, a treatment history comprising one or more historical treatments; and
determining, based on the treatment history, an error rate, wherein the error rate comprises a difference between the one or more programmatic treatments and the one or more historical treatments.

2. The method of claim 1, wherein the patient data comprises at least one of: a survey or an electronic health record.

3. The method of claim 1, wherein the care decision logic is associated with an identifier and wherein the identifier is associated with an ICD code.

4. The method of claim 1, wherein determining the care decision logic comprises:
receiving a user input; and
determining, based on the user input, the decision logic.

5. The method of claim 1, wherein determining the decision logic comprises:
determining, based on the one or more inputs, metadata associated with the input; and
selecting, based on the metadata, and from a database, the decision logic.

6. The method of claim 1, further comprising:
determining the error rate satisfies an error rate threshold; and
outputting, based on the error rate satisfying the error rate threshold, a message.

7. The method of claim 1, further comprising:
storing, in a database, a first record associated with the one or more programmatic diagnostic actions:
storing, in the database, a second record associated with the one or more historical diagnostic actions:
storing, in the database, the error rate; and
sending, to an external computing device, the first record associated with the one or more programmatic diagnostic actions, the second record associated with the one or more historical diagnostic actions, and the error rate.

8. A system comprising:
a computing device configured to:
receive, by a computing device, patient data:
determine, based on the patient data, a care decision logic:
execute, based on the patient data, the care decision logic to determine a care conclusion:
determine, based on executing the care decision logic and the care conclusion, one or more programmatic treatments:
receive, by the computing device, a treatment history comprising one or more historical treatments:
determine, based on the treatment history, an error rate, wherein the error rate comprises a difference between the one or more programmatic treatments and the one or more historical treatments; and
a display device configured to:
output the care conclusion.

9. The system of claim 8, wherein the patient data comprises at least one of: a survey or an electronic health record.

10. The system of claim 8, wherein the care decision logic is associated with an identifier and wherein the identifier is associated with a condition.

11. The system of claim 8, wherein the computing device configured to determine the care decision logic is further configured to:
receive a user input; and
determine, based on the user input, the care decision logic.

12. The system of claim 8, wherein the computing device configured to determine the care decision logic is further configured to:
determining, based on the one or more inputs, metadata associated with the input; and
selecting, based on the metadata, and from a database, the decision logic.

13. The system of claim 8, wherein the computing device configured to determine the care decision logic is further configured to:
determine the error rate satisfies an error rate threshold; and
output, based on the error rate satisfying the error rate threshold, a message.

14. The system of claim 8, wherein the computing device configured to determine the care decision logic is further configured to:
store in a database, a first record associated with the one or more programmatic diagnostic actions:
store, in the database, a second record associated with the one or more historical diagnostic actions:
store, in the database, the error rate; and
send, to an external computing device, the first record associated with the one or more programmatic diagnostic actions, the second record associated with the one or more historical diagnostic actions, and the error rate.

15. An apparatus, comprising:
one or more processors; and
memory storing processor executable instructions that, when executed by the one or more processors, cause the apparatus to:
receive, by a computing device, patient data:
determine, based on the patient data, a care decision logic:
execute, based on the patient data, the care decision logic to determine a care conclusion:
determine, based on executing the care decision logic and the care conclusion, one or more programmatic treatments or care decisions:
receive, by the computing device, a treatment or care decision history comprising one or more historical treatments or care decisions; and
determine, based on the treatment history historical care decisions, an error rate, wherein the error rate comprises a difference between the one or more programmatic treatments and the one or more historical treatments.

16. The apparatus of claim 15, wherein the patient data comprises at least one of: a survey or an electronic health record.

17. The apparatus of claim 15, wherein the care decision logic is associated with an identifier and wherein the identifier is associated with a condition.

18. The apparatus of claim 15, wherein the processor executable instructions, when executed by the one or more processors, further cause the apparatus to:
receive a user input; and
determine, based on the user input, the care decision logic.

19. The apparatus of claim 15, wherein the processor executable instructions, when executed by the one or more processors, further cause the apparatus to:
determine the error rate satisfies an error rate threshold; and
output, based on the error rate satisfying the error rate threshold, a message.

20. The apparatus of claim 15, wherein the processor executable instructions, when executed by the one or more processors, further cause the apparatus to:
store in a database, a first record associated with the one or more programmatic diagnostic actions:
store, in the database, a second record associated with the one or more historical diagnostic actions:
store, in the database, the error rate; and
send, to an external computing device, the first record associated with the one or more programmatic diagnostic actions, the second record associated with the one or more historical diagnostic actions, and the error rate.
